(19) United States
(12) Patent Application Publication
(10) Pub. No.: US 2013/0232639 A1
(43) Pub. Date: Sep. 5, 2013

(54) SONICATION-ASSISTED POLLEN-MEDIATED PLANT TRANSFORMATION METHOD

(71) Applicant: BIOTECHNOLOGY RESEARCH CENTER, SHANXI ACADEMY OF AGRICULTURAL SCIENCES, (US)

(72) Inventors: Yi SUN, Taiyuan (CN); Guimei CUI, Taiyuan (CN); Yaoshan HAO, Taiyuan (CN); Jianzhong DU, Taiyuan (CN); Yixue WANG, Taiyuan (CN); Liyan YANG, Taiyuan (CN); Hongmei ZHANG, Taiyuan (CN); Lijun ZHANG, Taiyuan (CN); Ming WANG, Taiyuan (CN); Tingting ZHANG, Taiyuan (CN)

(73) Assignee: BIOTECHNOLOGY RESEARCH CENTER, SHANXI ACADEMY OF AGRICULTURAL SCIENCES, Taiyuan (CN)

(21) Appl. No.: 13/863,366

(22) Filed: Apr. 15, 2013

Related U.S. Application Data
(63) Continuation-in-part of application No. PCT/CN2012/000030, filed on Jan. 9, 2012.

(30) Foreign Application Priority Data
Feb. 18, 2011 (CN) 201110041484.0

(51) Int. Cl. C12N 15/82

(52) U.S. Cl. CPC C12N 15/8206
USPC 800/270

(57) ABSTRACT

A transformation method, including the following steps: preparing an Agrobacterium Ti-plasmid, Escherichia coli plasmid, or other DNA vectors carrying exogenous genetic fragments as a genetic donor; collecting a male gamete (pollen) of the plant as a recipient; preparing a 5-50% sucrose solution after aeration and low temperature pretreatment; mixing the pollen with the exogenous genetic fragments in the 5-50% sucrose solution; transferring the exogenous genetic fragments into the pollen in the presence of ultrasonication; pollinating a pistil stigma of the plant with the treated pollen; harvesting seeds at maturity; sowing the seeds in a subsequent growing season; screening a germinating seed and a seedling; and performing PCR amplification and Southern hybridization using DNA samples of plants to further determine transformants.

### CROSS-REFERENCE TO RELATED APPLICATIONS

This application is a continuation-in-part of International Patent Application No. PCT/CN2012/000030 with an international filing date of Jan. 9, 2012, designating the United States, now pending, and further claims priority benefits to Chinese Patent Application No. 201110041484.0 filed Feb. 18, 2011. The contents of all of the aforementioned applications, including any intervening amendments thereto, are incorporated herein by reference. Inquiries from the public to applicants or assignees concerning this document or the related applications should be directed to: Matthias Scholl P. C., Attn.: Dr. Matthias Scholl Esq., 14781 Memorial Drive, Suite 1319, Houston, Tex. 77079.

### BACKGROUND OF THE INVENTION

1. Field of the Invention

The invention relates to a plant transformation method.

2. Description of the Related Art

At present, two classical methods adopted in plant transformation research are an Agrobacterium-mediated transformation method, and a particle bombardment method. Both methods require a long and complicated plant tissue culture process and are laborious, costly, and time-consuming As some plant species or varieties are difficult to generate by tissue culture, thus, both methods are highly genotype-dependent, the applications of these two methods have been seriously restricted. Furthermore, somatic variation and death of regenerated plantlets frequently occur during plant tissue culture process, thus the already low transformation ratio would be further reduced. All these defects have greatly limited the wide application of plant transformation technology. Due to the complexity to operate or low efficiency, other plant transformation methods are rarely used in practice despite the reports on their successful transformation. These methods include those involving liposome, PEG, electroporation, microinjection, ultrasonication, ion beam, laser microbeam puncture, and silicon carbide fiber. Therefore, an efficient and simplified plant transformation method has been sought.

A pollen-tube-pathway method has been applied to a certain extent, and some transgenic lines or varieties have been generated. This method is advantageous in no dependency on tissue culture or plant regeneration, no requirement on well-equipped labs, and its simplicity to operate. However, the transformation efficiency of this method is low and it requires selecting transformants among a large number of progenies. Therefore, the lack of a simple and efficient plant transformation method is still a bottleneck in plant genetic engineering. In a sonication-assisted pollen-mediated plant transformation method, a sonifier cell disrupter is used to treat pollen suspension by 200-300 W ultrasonication power. Fresh pollen is collected and suspended in a 5-15% sucrose solution including foreign DNA having a concentration of at least 40 μg/L. The pollen suspension is treated by ultrasonication, before and after the addition of the foreign DNA, for 5-8 times at an interval of 10 seconds, each for 5 seconds. Pollen is then pollinated to plant stigmas (silks in case of maize), seeds are harvested and sowed in the subsequent season, and transformants are selected from progenies. The method does not require a long and complicated tissue culture process, and is simple, effective, fast, economical, and thus highly practical. This method can be readily integrated into conventional breeding programs and directly used by crop breeders. However, a major shortcoming of this method is its low seed setting rate after pollination, because most pollen grains lose their viability after ultrasonication and fail to complete the fertilization process. Therefore, improving the seed setting rate is the key to a wider application prospect of the method.

### SUMMARY OF THE INVENTION

In view of the above-described problems, it is one objective of the invention to provide a sonication-assisted pollen-mediated plant transformation method that remarkably improves a seed setting rate of pollinated plants.

It is found experimentally that a higher proportion of pollen grains are capable of maintaining their vitality in well aerated sucrose solution at low temperature during pollen treatment, so that a higher seed setting rate is reached after the treated pollen is applied to plant stigmas (corn silks herein). Furthermore, it is favorable to enhance the pollen vitality by preserving newly collected pollen at a low temperature (4° C.) and under dry condition, and using the pollen within 2-48 h.

The method uses an Agrobacterium Ti-plasmid, an Escherichia coli plasmid, or other DNA vectors carrying an exogenous genetic fragment as a gene donor; uses male gametes of the plant as intermediate recipients, and the sonication-assisted pollen-mediated gene transfer is achieved in the plant pollination and fertilization process. Under actions of instantaneous high energy release and cavitations of ultrasonication, the foreign DNA fragments are introduced into the plant pollen, then enter plant female gamete embryo sacs along with a growth of pollen tubes to participate the formation of zygotes, and finally incorporated into the target plant genomes.

Specific process is as follows: a 5-50% sucrose solution is aerated and low temperature treated, and fresh pollen is mixed with the foreign DNA in the 5-50% sucrose solution. The foreign DNA fragments are introduced into the pollen by ultrasonication. The treated pollen is pollinated to plant stigmas. Seeds are harvested at maturity, and then sowed in the subsequent growing season. Transformants are further determined through screening germinating seeds and seedlings with a selector (often but not limited to a herbicide or an antibiotic) and PCR amplification and Southern hybridization on plant DNA samples.

Before the addition of the pollen and the foreign DNA, the sucrose solution is pretreated with aeration and ice bath. The solution is continuously aerated for more than 20 minutes by a miniature commercially-available aquarium air pump until the air (oxygen) content in the sucrose solution is saturated. Meanwhile, the solution is placed in a 0-4° C. ice bath or a refrigerator. The pollen suspension is always placed in the 0-4° C. ice bath during subsequent operations. The ultrasonic treatment is applied to the pollen suspension before or after the addition of the foreign DNA, with the power of the ultrasonication of 50-500 W, and the time of the ultrasonic treatment of 5 seconds to 2 minutes.

The fresh pollen can be preserved for 5 days at a temperature of 4° C. with certain pollen viability. The treated pollen is pollinated to the plant stigma, and seeds on the pollinated ears are harvested at the maturity and then sowed in the subsequent growing period. The transformants are determined through screening of the seedlings (the step can be skipped in a marker-free transformation), and PCR amplification, and Southern hybridization of genomic DNA. The transformant is continuously self-pollinated and selected until a stable and homozygous transgenic line is obtained.

The improved pollen-mediated plant transformation method assisted by ultrasonication is capable to markedly improve seed setting rate following pollination, thereby to increase the overall transformation efficiency. In the method, the exogenous gene can be directly transferred into the recipient genome, thus the complicated plant tissue culture process with demanding technical operation is avoided, and the turnaround time to obtain the transformed seeds is greatly shortened. The method has the merits of high gene transformation efficiency, good reproducibility, less probability of chimera plants, cheap operational requirement, and genotype independency (i.e., wide range of application). Furthermore, this method can be applied to high-throughput transformation systems as it improves the seed setting rate and thus the transformation efficiency.

### DETAILED DESCRIPTION OF THE EMBODIMENTS

For further illustrating the invention, experiments detailing an improved plant transformation method applied in corns are described below. It should be noted that the following examples are intended to describe and not to limit the invention.

Except for unavoidable damages under the application of ultrasonication, the cause of low pollen vitality due to the treatment lies partially to the damage of pollen in the suspension, in cases of pollen breakage, and plasmolysis.

The vitality of pollen can be improved by the modification of the suspension conditions of pollen, of which, sucrose concentration (osmotic pressure), temperature, and air (oxygen) content are three main factors. In view of the above factors, the following experiments are made with the corn variety Zheng 58, and the results are shown in Tables 1-8.

### EXAMPLE 1

### Significant Influence of Soaking Time of Corn Pollen in Suspension on In Vitro Germination

As shown in Table 1, with the extension of the soaking time, the broken rate of corn pollen was increased, and the germination rate was remarkably reduced. After the corn pollen was soaked for 1 hour in suspension without aeration treatment at the temperature of 28° C., the germination rate was reduced to 20%. If the soaking time reached 120 minutes, the pollen germination rate was close to zero.

TABLE 1
Reduction of In vitro germination rate of corn pollen with the increase of
soaking time in the suspension

| | Soaking time | | | | | | | | |
|---|---|---|---|---|---|---|---|---|---|
| | 0 min | 10 min | 20 min | 30 min | 40 min | 50 min | 60 min | 90 min | 120 min |
| Broken rate of | 12.8 ± 2.29f | 18.6 ± 2.88f | 15.7 ± 3.10f | 28.2 ± 4.81e | 42.3 ± 5.57d | 51.1 ± 6.37c | 56.3 ± 6.85bc | 63.8 ± 6.84b | 78.9 ± 6.45a |
| pollen (%) | | | | | | | | | |
| Germination rate | 81.4 ± 5.52a | 78.3 ± 5.76a | 75.4 ± 5.23a | 65.2 ± 6.18b | 48.3 ± 5.97c | 32.4 ± 5.54d | 20.8 ± 4.17e | 15.7 ± 3.26e | 0.16 ± 0.22f |
| of pollen (%) | | | | | | | | | |

Note:
the pollen germination rate was determined after soaking the pollen in a 15% sucrose solution at the room temperature of 28° C. by the incubation of pollen suspension into a culture medium for 30 minutes. The medium was prepared as follow: 15% sucrose + 50 mg/L boric acid + 300 mg/L calcium chloride + 200 mg/L magnesium chloride + 100 mg/L potassium nitrate + 35 mg/L gibberellin.
Means not sharing the same letters indicate significant difference (P < 0.05).

### EXAMPLE 2

### Sucrose Concentration Plays an Important Role in Pollen Germination Rate

The sucrose was mainly used as an osmotic agent in the solution. As shown in Table 2-1 and Table 2-2, an average breakage rate of corn pollen in the sucrose solution of a low concentration (≦5%) was higher when pollen incubation was carried out at any time. The rate of undamaged pollen was increased with the increase of the sucrose concentration. However, when the concentration of the sucrose was reached up to 50%, the pollen germination rate was remarkably reduced.

TABLE 2-1
The pollen viability following the In vitro incubation in the culture media
with different sucrose concentrations (Greenhouse trial)

| | | Germination | | |
|---|---|---|---|---|
| Sucrose | Broken rate of | rate of pollen | Length of pollen | |
| concentration | pollen (%) | (%) | tube (μm) | Characteristics |
| 1% | 72.5 ± 6.85a | 7.26 ± 2.37e | 200 ± 42.3f | A large amount of pollen |
| | | | | was broken with internal |
| | | | | content leaking out into |
| | | | | the medium; pollen tubes |
| | | | | were short and slender |
| 5% | 60.3 ± 6.07b | 11.1 ± 4.88e | 262 ± 48.1f | Same as above |
| 10% | 32.9 ± 4.76c | 56.5 ± 5.69c | 671 ± 50.2d | Some pollen tubes were |
| | | | | stretched and broke. |
| 15% | 30.8 ± 4.11cd | 60.0 ± 5.82bc | 1357 ± 58.4c | Same as above, pollen |
| | | | | tubes were relatively |
| | | | | longer. |
| 20% | 26.6 ± 3.73cde | 71.2 ± 4.71a | 1804 ± 68.7b | Pollen breakage was |
| | | | | reduced; pollen tubes |
| | | | | were relatively long, grew |
| | | | | smooth, straight, and |
| | | | | evenly. |
| 30% | 25.1 ± 3.51def | 65.6 ± 5.63ab | 2058 ± 62.4a | Same as above, pollen |
| | | | | tubes were the longest, |
| | | | | and grew normally. |
| 40% | 22.4 ± 3.71ef | 42.3 ± 5.38d | 756 ± 51.7d | Internal content |
| | | | | aggregated after pollen |
| | | | | was broken, pollen tubes |
| | | | | were relatively short. |
| 50% | 17.9 ± 3.24f | 12.2 ± 4.61e | 380 ± 45.9e | Pollen was seldom |
| | | | | broken, internal content |
| | | | | aggregated; pollen tubes |
| | | | | were chunky with some of |
| | | | | them deformed. |

TABLE 2-2
The pollen viability following the in vitro incubation in the culture media
with different sucrose concentrations (field trial)

| | | Germination | | |
|---|---|---|---|---|
| Sucrose | Broken rate of | rat of pollen | Length of pollen | |
| concentration | pollen (%) | (%) | tube (μm) | Characteristics |
| 1% | 66.4 ± 6.08a | 8.69 ± 2.12ef | 703 ± 54.2e | A large amount of pollen was |
| | | | | broken with internal content |
| | | | | leaking out into the medium; |
| | | | | pollen tubes were short with a |
| | | | | tip easily broken |
| 5% | 51.9 ± 5.88b | 17.8 ± 5.47d | 1428 ± 72.5c | Although the broken rate was |
| | | | | high, and the germination rate |
| | | | | was low, pollen tubes were |
| | | | | long and well grown. |
| 10% | 40.1 ± 5.24c | 67.4 ± 5.72b | 2206 ± 78.9b | Germination rate was high; |
| | | | | pollen tubes grew well, and |
| | | | | were smooth and even. |
| 15% | 23.8 ± 3.83d | 80.6 ± 4.94a | 2625 ± 65.5a | Broken rate was the lowest, |
| | | | | germination rate was the |
| | | | | highest; and the pollen tubes |
| | | | | were the longest. |
| 20% | 11.4 ± 2.10e | 49.6 ± 5.12c | 2285 ± 62.0b | Pollen broken rate was sharply |
| | | | | declined, and the pollen grew |
| | | | | normally. |
| 30% | 4.07 ± 1.17f | 12.8 ± 5.02de | 1188 ± 57.6d | Same as above, internal content |
| | | | | aggregated after the pollen was |
| | | | | broken. |
| 40% | 2.91 ± 1.00f | 2.37 ± 1.21f | 200 ± 46.3f | Pollen broken rate was low, |
| | | | | internal content was |
| | | | | filamentous, and pollen tubes |
| | | | | were chunky. |
| 50% | 0.50 ± 0.47f | 0 | 0 | Pollen was rarely broken; |
| | | | | bubble like black circles |
| | | | | appeared inside the pollen; and |
| | | | | no germination. |

From the comparison of in vitro incubation of corn pollen under different growing conditions, the pollen viability from plants grown in different phenological conditions reacted differently to same sucrose concentration. Early-sowed corn was sowed in a greenhouse on Mar. 29, 2010, and pollen was collected from May 28 to June 10. Field corn was sowed in an isolated experimental plot on April 29, and pollen was collected from July 15 to August 5. The study was carried out in Taiyuan, Shanxi, China. As shown in Table 2-1 and Table 2-2, the optimal sucrose concentration for the germination of greenhouse pollen was 20%-30%, and pollen germination was still observed in the 50% sucrose solution although in a rather low rate. the optimal sucrose concentration for field pollen germination was 15%, the sucrose concentration higher than 20% inhibited the germination, and pollen plasmolysis appeared in the 50% sucrose solution which resulted in little pollen germination. Compared with the greenhouse corn, the field corn had lower pollen broken rate in the same sucrose concentration, and has longer length and faster growth of the pollen tube at the same germination rate.

In conclusion, the lower-concentration sucrose is preferably used as the culture medium for pollen germination of the field-grown corn in Taiyuan, Shanxi China; the higher sucrose concentration is appropriate for pollen germination of corn sowed in greenhouse or other phenological conditions of low temperature and humidity.

### EXAMPLE 3

### Effects of Preservation Time and Conditions on Pollen Viability

Viability of pollen increases in the order in conditions of room temperature humid (25-28° C., RH 50-70%), room temperature dry (25-28° C., RH 30-50%), low temperature humid (10-15° C., RH 70-90%), and low temperature dry (4° C., RH 40-60%). Particularly, pollen germination rate was favorably preserved when the pollen was kept in a Petri dish containing culture medium at 4° C., which is ideal for the sonication-mediated plant transformation. As shown in Table 3-1, the in vitro living time of greenhouse pollen was only 2 h at low temperature and in dry condition; thereafter, the pollen germination rate was reduced substantially, and thus, the greenhouse corn pollen was less ideal. In a Petri dish at 4° C., the field pollen was still viable at a low rate even (Table 3-2) after 5 days' dry preservation. The newly collected pollen was very easy to be broken and had a low germination rate; however, the germination rate was substantially improved after 2 hours' preservation at dry and low temperature conditions, and the pollen had high vitality within 48 hours. The germination rate and the preservation time of corn pollen were related with the quality of the pollen collected on the day. In conclusion, the field corn pollen collected in the normal growing season under good phenological conditions had high tolerance, low pollen broken rate, and fast growth of pollen tube.

TABLE 3-1
In vitro germination results of the early-sowed corn under various
preservation time and preservation conditions (%)

| Preservation | Time for collecting pollen | | |
|---|---|---|---|
| condition and time | 8:30 | 10:00 | 11:30 |
| Immediate | 18.3 ± 4.33a | 56.9 ± 6.72a | 69.2 ± 6.79a |
| germination | | | |
| Low temperature | 7.30 ± 2.14b | 42.2 ± 5.88b | 68.8 ± 6.45a |
| dying for 2 h | | | |
| Low temperature | 0 | 15.2 ± 4.13c | 35.4 ± 5.62b |
| humid for 2 h | | | |
| Room temperature | 0 | 9.3 ± 3.02cd | 12.5 ± 3.67c |
| drying for 2 h | | | |
| Room temperature | 0 | 0 | 0 |
| humid for 2 h | | | |
| Low temperature | 0 | 5.2 ± 2.13d | 8.26 ± 2.75c |
| drying for 4 h | | | |
| Low temperature | 0 | 0 | 0 |
| drying for 6 h | | | |

TABLE 3-2
In vitro germination of pollen from field-grown corn at 4° C. dry preservation

| | Preservation time | | | | | | | | | |
|---|---|---|---|---|---|---|---|---|---|---|
| | 0.5 h | 2 h | 4 h | 6 h | 24 h | 48 h | 72 h | 96 h | 120 h | 144 h |
| Pollen broken | 48.2 ± | 26.6 ± | 21.5 ± | 18.4 ± | 16.7 ± 2.55e | 24.1 ± 3.34de | 46.3 ± 5.15c | 52.8 ± 6.28c | 79.8 ± 6.91b | 90.4 ± |
| rate (%) | 5.29c | 3.86d | 3.17de | 2.81de | | | | | | 7.87a |
| Pollen | 35.0 ± | 68.3 ± | 75.6 ± | 80.2 ± | 84.3 ± 5.27a | 72.4 ± 5.94bc | 45.8 ± 4.57d | 35.7 ± 4.16e | 15.9 ± 3.27f | 0 |
| germination rate | 4.52e | 5.75c | 5.43bc | 5.18ab | | | | | | |
| (%) | | | | | | | | | | |
| Length of poller | 2000 ± | 2250 ± | 2500 ± | 2750 ± | 3000 ± 78.5a | 1500 ± 72.3f | 1000 ± 68.4g | 625 ± 52.8h | 250 ± 41.2i | 0 |
| tube (μm) | 71.6e | 76.5d | 73.8c | 74.2b | | | | | | |

### EXAMPLE 4

### Effects of Suspension Temperature on Pollen Germination

The temperature of the pollen suspension affected the pollen germination, and low temperature reduced pollen breakage.

Note: the germination rate was determined after soaking the pollen in the 15% sucrose solution for 5 minutes at a proper temperature, and a few droplets of pollen suspension were transferred and incubated in culture medium to measure the germination rate.

TABLE 4
Pollen germination in suspension solution at various temperatures

| | temperature | | | | | | |
|---|---|---|---|---|---|---|---|
| | 35° C. | 30° C. | 25° C. | 20° C. | 15° C. | 10° C. | 4° C. |
| Pollen broken | 32.2 ± 5.26a | 22.4 ± 3.89b | 18.5 ± 2.87bc | 18.4 ± 2.56bc | 16.7 ± 2.23cd | 15.1 ± 2.75cd | 13.3 ± 2.15d |
| rate (%) | | | | | | | |
| Pollen | 64.3 ± 4.66b | 76.3 ± 5.19a | 78.2 ± 5.31a | 74.3 ± 5.07a | 79.7 ± 5.44a | 73.4 ± 5.47a | 75.8 ± 5.58a |
| germination | | | | | | | |
| rate* (%) | | | | | | | |

### EXAMPLE 5

### Effects of Solution Aeration on Pollen Germination

It should be noted that during the pollen-mediated plant transformation operation, a rapid germination of pollen in the suspension is not favorable for improving the seed setting rate and the transformation ratio, because the germinated pollen tube would probably be damaged during a subsequent pollination, and it is not easy for the germinated pollen tube to grow into the stigma and to complete fertilization. Under the ideal state, the pollen does not germinate or break in the suspension, and its vitality is maintained. Therefore, both pollen germination rate and fertilization rate will be high following pollination. As shown in Table 5, the germination rate was low, and so was the broken rate when pollen suspension was subjected to 20 minutes' aeration treatment.

TABLE 5
Effect of suspension aeration on pollen germination*

| | Aeration time | | | | | | |
|---|---|---|---|---|---|---|---|
| | 0 min | 10 min | 20 min | 30 min | 40 min | 50 min | 60 min |
| Pollen broken | 27.2 ± 4.52a | 21.7 ± 3.77b | 16.5 ± 2.45c | 18.4 ± 2.66bc | 14.7 ± 2.13c | 16.4 ± 2.73c | 13.8 ± 2.24c |
| rate (%) | | | | | | | |
| Pollen | 78.4 ± 4.68a | 56.3 ± 4.26b | 45.1 ± 3.78c | 51.4 ± 3.85b | 42.7 ± 3.71c | 43.5 ± 2.86c | 45.3 ± 3.18c |
| germination | | | | | | | |
| rate* (%) | | | | | | | |

Note:
germination rate was determined by suspending the pollen in the 15% sucrose solution (aerated for at least 20 minutes) for 5 minutes at the temperature of 25° C. and a few droplets of pollen suspension being transferred to culture medium for the measurement.

### EXAMPLE 6

### The Effects of Temperature and Aeration on Pollen Germination After Ultrasonication

Ultrasonication was a key step for the exogenous gene(s) to enter the pollen. The experiment (Table 6) showed that the aeration and low temperature treatment remarkably reduced the pollen broken rate and improved pollen germination rate after ultrasonication. The 11.9% of aeration-treated pollen was able to germinate compared to 3.74% of the untreated control.

TABLE 6
Pollen germination in various pretreated sucrose solution before and after
ultrasonication*

| | Pollen before ultrasonication | | | Pollen after ultrasonication | | |
|---|---|---|---|---|---|---|
| Pretreatments | | Germination | | | Germination | |
| on sucrose | Broken | rate | Length of pollen | Broken | rate | Length of pollen |
| solution | rate (%) | (%) | tube (μm) | rate (%) | (%) | tube (μm) |
| Room | 43.1 ± 5.11a | 62.7 ± 3.43a | 2843.8 ± 146.11a | 80.1 ± 8.15a | 3.74 ± 1.22c | 821.88 ± 100.39c |
| temperature | | | | | | |
| 28° C. | | | | | | |
| Low | 30.7 ± 4.01b | 66.4 ± 4.05a | 2915.6 ± 151.74a | 61.0 ± 6.85b | 8.19 ± 1.36b | 1378.1 ± 136.56ab |
| temperature | | | | | | |
| 4° C. | | | | | | |
| Aerating for | 15.4 ± 2.81c | 42.1 ± 3.65b | 2431.3 ± 143.15b | 32.5 ± 4.16c | 6.03 ± 1.23b | 1221.9 ± 112.95b |
| 20 min | | | | | | |
| Aerating for | 13.5 ± 2.49c | 40.6 ± 3.67b | 2450.0 ± 136.93b | 24.5 ± 3.37c | 11.9 ± 2.39a | 1528.1 ± 150.26a |
| 20 min at a | | | | | | |
| low | | | | | | |
| temperature | | | | | | |
| of 4° C. | | | | | | |

Note:
the pollen was suspended in the 15% sucrose solution of various pretreatments for 5 minutes prior to ultrasonication, and then a few droplets of pollen suspension were transferred to culture medium to germinate. For the germination rate measurement after ultrasonication, a few droplets of pollen were immediately transferred to the culture medium following the ultrasonication treatment.

### EXAMPLE 7

### Effects of Various Pollen Treatments on Seed Setting Rate After Pollination

Pretreated pollen was pollinated to corn silks, and seed setting rates were recorded. As shown in Table 7, aeration treatment was more important than that of low temperature treatment; the combination of aeration and low temperature was the most favorable treatment for seed setting; and the average seed set per ear was improved by 1.27 times (1.65: 0.728).

TABLE 7
Effects of different treatment on seed setting rate after pollination

| | | | | Number of | | | Seed setting ear | | | | Seed number per |
|---|---|---|---|---|---|---|---|---|---|---|---|
| | Number of | | | seed setting | | | rate (B/A)% | | | | ear (C/A) |
| Treatment | treated ears (A) | | | ears (B) | | | Means ± SD | Seed number (C) | | | means ± SD |
| Ultrasonication | 27 | 27 | 27 | 3 | 4 | 4 | 13.58 ± 1.14b | 17 | 19 | 23 | 0.728 ± 0.113c |
| Ultrasonication + | 113 | 113 | 113 | 15 | 17 | 19 | 15.04 ± 1.77ab | 96 | 99 | 87 | 0.832 ± 0.055c |
| low | | | | | | | | | | | |
| temperature | | | | | | | | | | | |
| Ultrasonication + | 315 | 315 | 315 | 51 | 60 | 64 | 18.52 ± 2.15a | 392 | 401 | 447 | 1.31 ± 0.096b |
| aeration | | | | | | | | | | | |
| Ultrasonication + | 280 | 280 | 280 | 40 | 41 | 45 | 15.0 ± 2.45ab | 439 | 451 | 495 | 1.65 ± 0.106a |
| low | | | | | | | | | | | |
| temperature + | | | | | | | | | | | |
| aeration | | | | | | | | | | | |

### EXAMPLE 8

### Transformation Ratio by Various Pollen Pretreatments

A transformation vector carrying a bar gene was employed in the transformation, which was capable to make transformants to resist the herbicide basta. Therefore, the transformants were preliminarily screened by spraying the herbicide. Corn seeds after transformation were sowed in plots, and sprayed with 2% herbicide basta at a 5-6 leaf stage. Refer to Table 8 for results of herbicide screening for genetically modified seedlings.

TABLE 8
Herbicide resistant rate after pollination of different treatments of pollen

| | | Seeding | Seeding |
|---|---|---|---|
| | | emergence | emergence |
| Treatment | Sowing number | number | rate (%) |
| Ultrasonication | 59 (19, 20, 20) | 51 (15, 17, 19 | 86.44b |
| Ultrasonication + | 215 (70, 72, 73) | 186 (58, 63, 65) | 86.51b |
| low temperature | | | |
| Ultrasonication + | 222 (76, 80, 66) | 197 (68, 69, 60) | 88.74b |
| aeration | | | |
| Ultrasonication + | 315 (93, 110, 112) | 263 (76, 95, 92) | 83.49b |
| aeration + low | | | |
| temperature | | | |
| CK (untransformed) | 91 (30, 30, 31) | 91 (30, 31, 31) | 100a |

| | | Herbicide | | |
|---|---|---|---|---|
| | Herbicide | resistant | PCR | PCR |
| | resistant | strain rate | positive | positive |
| Treatment | strain | (%) | strain | strain rate |
| Ultrasonication | 25 (8, 11, 6) | 49.02a | 10 | 19.6a |
| Ultrasonication + | 97 (32, 30, 35) | 52.15a | 36 | 19.4a |
| low | | | | |
| temperature | | | | |
| Ultrasonication + | 103 (33, 38, 32) | 52.28a | 41 | 20.8a |
| aeration | | | | |
| Ultrasonication + | 134 (40, 51, 43) | 51.0a | 55 | 20.9a |
| aeration + low | | | | |
| temperature | | | | |
| CK | 0 | 0b | | |
| (untransformed) | | | | |

Note:
the resistant plant rate was the number of herbicide resistant plants divided by the number of total seedlings. The PCR positive plant rate was the number of PCR positive plants divided by the number of total seedlings.

As shown in Table 8, T₀-generation seeds obtained after various pretreatments did not show significant difference on the ratio of the herbicide resistant plants. Leaves of individual herbicide resistant plants were collected at the 5-leaf stage and total DNA was extracted for PCR analysis. The results showed that about 20% PCR positive plants were obtained no matter which method was used for pollen treatment. Southern hybridization confirmed that all the PCR positive plants were transformants, which indicates that the exogenous gene had been introduced into the recipient plants.

The above results indicated that the improved method did not produce a significant influence on the transformation ratio while remarkably improved the seed setting rate after pollination, therefore, the number of transformants obtained from each pollination was enhanced remarkably.

While particular embodiments of the invention have been shown and described, it will be obvious to those skilled in the art that changes and modifications may be made without departing from the invention in its broader aspects, and therefore, the aim in the appended claims is to cover all such changes and modifications as fall within the true spirit and scope of the invention.

The invention claimed is:

1. A plant transformation method, the method comprising the following steps:
a) preparing an Agrobacterium Ti-plasmid, an Escherichia coli plasmid, or other DNA vectors carrying an exogenous genetic fragment as a genetic donor;
b) collecting a male gamete (pollen) of a plant as an intermediate-recipient;
c) preparing a 5-50% sucrose solution after aeration and low temperature pretreatment;
d) mixing the pollen with the exogenous genetic fragment in the 5-50% sucrose solution;
e) transferring the exogenous genetic fragment to the pollen in the presence of ultrasonication;
f) pollinating a pistil stigma of the plant with the treated pollen;
g) harvesting a seed at maturity;
h) sowing the seed in a subsequent growing season;
i) screening a germinating seed and a seedling; and
j) performing PCR amplification and a Southern hybridization using a DNA sample of an adult plant for further determination of a transformant.

2. The method of claim 1, wherein the pollen is fresh or preserved within 5 days at a temperature of 4° C.

3. The method of claim 1, wherein
the aeration and low temperature pretreatment of the sucrose solution before the addition of the pollen and foreign DNA fragment are as follows: continuously aerating the air into the sucrose solution for more than 20 minutes using an air pump until an air content in the sucrose solution is saturated; while placing the sucrose solution in a 0-4° C. ice bath or a refrigerator for pretreatment; and a pollen suspension solution is placed in a 0-4° C. ice bath for additional steps.

4. The method of claim 3, wherein
the ultrasonication is performed on the pollen suspension before and after the addition of the foreign DNA fragment;
a power of the ultrasonication is 50-500 W; and
a duration of the ultrasonic treatment is 5 seconds to 2 minutes.

5. The method of claim 1, wherein the determination of the germinating seed and the seedling is achieved by a selector according to a selective marker gene of the recipient.

6. The method of claim 1, wherein the determination of the adult plant is achieved by the PCR amplification and the Southern hybridization based on an inserted exogenous gene.

7. The method of claim 1, wherein the selected transformant is continuously self-pollinated and selected until a stable and homozygous transgenic line is obtained.

8. The method of claim 5, wherein the selector is an antibiotic or a herbicide.
